(19) United States
(12) Patent Application Publication
(10) Pub. No.: US 2006/0222614 A1
(43) Pub. Date: Oct. 5, 2006

(54) Hair straightening compositions and methods

(76) Inventor: Carol J. Buck, Princeton, NJ (US)

Correspondence Address: KAPLAN GILMAN GIBSON & DERNIER L.L.P., 900 ROUTE 9 NORTH, WOODBRIDGE, NJ, 07095, US

(21) Appl. No.: 11/098,658

(22) Filed: Apr. 4, 2005

(51) Int. Cl. A61K 8/73

(52) U.S. Cl. 424/70.7; 424/70.13

(57) ABSTRACT

Compositions and methods for the straightening and defrizzing of mammalian hair containing alkyl polyglucosides (APGs) in an amount at least 15% by weight of the composition. Compositions are preferably included in hair care products such as shampoo, conditioner, styling gel or hairspray.

### FIELD OF THE INVENTION

The present invention relates to compositions for treating mammalian hair and in particular to novel compositions containing alkyl polyglucosides (APGs) for the straightening, defrizzing, and smoothing of mammalian hair and methods of using same.

### BACKGROUND OF THE INVENTION

Current art for the straightening of mammalian hair involves the use of reducing agents such as thioglycolic acid, particularly as ammonium salt, to cleave the hair's cystine disulfide bonds, followed by the application of an oxidizing agent to stop the reaction while the hair is in the desired straight configuration. Such methods result in a dramatic reduction of curl and last for 6 to 8 weeks, but often cause the shaft to become brittle and, if the scalp is not carefully protected from the high alkalinity of these chemicals, follicle and epidermis injury can also result, especially after multiple applications.

U.S. Pat. No. 6,517,822 discloses alkanoic acid formulations, preferably acetic acid, to gently reduce curl and frizz in hair when coated on the shaft for several hours. The resultant loss of curl is less dramatic than conventional alkaline formulations but has the advantage of being non-caustic to the hair shaft and skin.

It would be advantageous to provide a mild hair straightening composition that is safe for the skin, and especially advantageous for users such as children having sensitive skin and/or short hair.

### SUMMARY OF THE INVENTION

The present invention relates to compositions and methods which gently and mildly relax curl and reduce frizz without damage to the cuticle or matrix of the hair shaft. The invention is also useful in removing unwanted curl or frizziness after a permanent wave treatment, as a refresher or “touch-up” to previously relaxed hair, or as a way to reduce curl and frizz that occurs only seasonally, due to changes in humidity with summer weather.

Compositions in accordance with the present invention contain alkyl polyglucosides (APG) present in mammalian hair care formulations in an amount of about 15% w/w and greater. The present inventor has found surprisingly that APGs in these amounts have a mild straightening effect on mammalian hair. Surprisingly, use of compositions in accordance with the present invention results in hair that is straighter, less frizzy, fuller in volume, easier to style and capable of retaining its style longer than hair treated with conventional formulations.

APGs are known to be environmentally safe and non-irritating to skin. APGs have strong anti-microbial properties with excellent viscosity and detergency as a cleanser. The mildness, foaming ability and ease of rinsing off that characterize APGs support their utility as an ingredient as mild non-ionic surfactants and foam stabilizers in shampoos and cosmetic cleansers calling for a non-ionic surfactant. In baby shampoos APGs serve to replace other more irritating surfactants, such as sodium lauryl sulfate, which is known to be absorbed through the eye and potentially damaging to the eyes of infants and children. However, the percent compositions of APGs (available commercially containing about 40-50% water) in hair care formulations are low, the formulations containing typically less than 10% w/w of such aqueous solutions of APGs . Despite the presence of APGs in personal care products, it has been heretofore unknown that APGs that are present in concentrations greater than 15% can have a hair straightening effect.

### DETAILED DESCRIPTION OF THE PREFERRED EMBODIMENTS

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

Compositions in accordance with the present invention contain one or more alkyl polyglucosides present in mammalian hair care formulations in amounts of at least about 15% w/w and greater.

Hair care formulations in accordance with the present invention may include shampoos, conditioners, gels, mousses, tonics, sprays and the like.

APGs customarily used for cosmetic and cleansing applications may be present in compositions according to at least one aspect of the present invention in amounts of from about 15% w/w and greater, preferably about 20% to about 90% w/w and most preferably about 40% to about 70% w/w. Commercially available APGs, such as but not limited to APG-C1214 (available from KW Chemicals, Hong Kong), Plantacare® 818 UP, Plantacare® 2000 UP, Plantaren® 1200 N UP and Plantaren® 2000N (all available from Cognis Corporation, New Milford, Conn., as well as, Suganate® 100 and Suganate® 160 (available from Colonial Chemical, Inc., South Pittsburg, Tenn.) may be used in compositions in accordance with the present invention. Those skilled in the art will recognize that these commercially available APGs in their purest form contain about 50-60% w/w water. For example, Suganate® 160 comprises approximately 60% w/w water. Compositions in accordance with the present invention may further contain cosmetic excipients such as but not limited to antibacterial agents, fragrance, rheology agents, conditioners, pearlizing agents, foam/viscosity stabilizers, co-surfactants, detanglers, color protectants, processing aids, dyes, foaming agents, water and pH adjusters.

In a preferred embodiment a composition in accordance with the present invention contains either one or both of sodium decylglucosides hydroxyproplylsulfonate (proposed CAS 742087-48-5), commercially available as Suga®Nate 100 by Colonial Chemical Corp. and sodium laurylglucosides hydroxypropyl sulfonate, commercially available as Suga® Nate 160 by Colonial Chemical Corp.

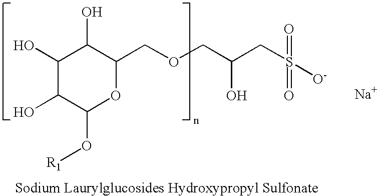

The gentleness of the present compositions to the hair and skin has the benefit of allowing the user to control exactly how much curl or frizz is to be removed. Importantly, the user need not be concerned about hair breakage or damage, as is the case with most fast-acting, alkaline hair-straightening formulations which relax hair by breaking disulfide bonds. The present compositions are safe enough for use on short hair and children's hair.

In a preferred embodiment topical, leave-in styling aids such as gels, serums, creams, mousses, etc. employ concentrations of APG of about 15-50% w/w. In another embodiment shampoos and wash-out styling aids such as conditioners employ concentrations of APG of about 30% w/w and greater. It will be apparent to those skilled in the art that the foregoing concentration ranges for these products are only preferred embodiments, and may be higher or lower in hair care products.

Formulations in accordance with the present invention have a pH in the range of from about 4.0 to about 11.5, most preferably in a range of from about 8.0 to about 10.0 and most preferably about 9.0. Those skilled in the art will recognize adjustment of pH to a final formulation may be made by suitable well known pH adjustors such as but not limited to citric acid to achieve a desired pH.

In a preferred embodiment a shampoo composition is provided in which at least one alkyl polyglucoside is diluted with water and blended with a washing base, such as sodium lauryl sulfate, and other excipients, such as a bactericide, fragrance, detangling agent, conditioner or color-retaining agent.

In an alternate embodiment, a hair conditioner composition is provided containing at least one hair conditioner such as d-panthenol, a polyquaternium, plant oils such as grapeseed oil, and excipients such as but not limited to rheology agents, emollients such as PEG 400 monostearate, colorants and fragrance.

Compositions in accordance with the present invention have a significant straightening and smoothing effect on human hair when applied in a conventional manner; that is, massaged into a low foam for three minutes and rinsed off (shampoo method) or applied to wet or dry hair with the palm and distributed evenly by combing or brushing (styling gel or conditioner method).

A method of using a shampoo composition according to at least one aspect of the present invention includes applying the composition to wet hair, massaging the composition into the hair for a selected length of time such as for example three minutes and rinsing the composition from the hair with water, preferably warm water. The method may further comprise a second application of shampoo especially if the treated hair has received previous applications of styling gel or sprays. In a preferred embodiment the method further comprises towel-drying the hair and pulling the hair straight to air-dry. Alternatively, the hair is blown dry using a hand held hair-dryer, or set on rollers and dried by natural and heated air under a bonnet hood-dryer. Faster results are achieved if the hair is pulled straight during drying. Electric appliances such as heat wands, electric curlers or flat-irons can be used to add shine and styling, but are not necessary to achieve relaxation of the hair.

A method of using a conditioner composition according to at least one aspect of the present invention includes applying the composition to wet hair, massaging the composition into the hair for a selected length of time and rinsing the composition from the hair with water, preferably warm water. In a preferred embodiment the step of applying the conditioner composition is performed after a shampoo treatment, preferably after a shampoo treatment employing a composition in accordance with the present invention.

A method of using a styling gel composition according to at least one aspect of the present invention includes applying the styling gel composition to hair. Preferably the composition is applied after washing the hair and after the hair is towel-dried and before air or blow-drying.

### EXPERIMENTS

Compositions in accordance with the present invention were tested on male and female subjects with curly and frizzy hair. Hair washed or conditioned with formulations described in the following Examples was significantly straighter after two to eight applications, with Caucasian hair responding more quickly, i.e., in fewer treatments, than African-American or Hispanic hair. Hair also remained straighter, more manageable and smoother (non-frizzy) for 3 to 8 days despite exposure to humidity (in the form of fog or morning mist). Treated hair was also observed to retain a smoother texture and style without requiring a wetting or resetting with heat-styling appliances, such as hot rollers or a flat-iron, which is usually done in one form or another by most men and women as part of their daily morning routine.

### Example Formula 1

Straightening/De-Frizzing Shampoo (2000 ml)

| Ingredient | % by weight |
|---|---|
| Deionized water | QS to 100% |
| APG 818 ® UP (Cognis) | 41.0% |
| Suttocide A (bactericide, ISP) | 1.0% |
| Fragrance | 1.0% |
| pH adjuster | QS to pH of 8.5-9.0 |

The formulation is prepared by charging the kettle with water, then adding the bactericide and fragrance. The APG is added to this mixture under slow agitation and blended for 30 minutes. The pH is then lowered to 8.5-9.0 by adding a pH adjuster such as aqueous acetic acid (12-20% solution) or citric acid and blended until homogeneous.

### Example Formula 2

Low-Foam/High-Foam Straightening/Defrizzing Shampoo with Color-Protectant and Detangler (2000 ml)

| Ingredient | % by weight |
|---|---|
| Deionized Water | QS to 100% |
| Alkyl Polyglucoside - SugaNate ® 160 UP* | 44.5 |
| (Colonial Chemical) | |
| Polyquaternarium - Merquat ® 2001 (Nalco) | 2.0 |
| Suttocide A | 0.5 |
| Fragrance | 0.5 |
| Citric Acid Anhydrous Powder (Spectrum) | QS to pH 9.0 |

This example, and all APG shampoos formulated without sodium lauryl/laureth sulfate or other foaming agent, typically produce a low lather with the first application and a high lather with the second application. Shampoos in accordance with this formulation are preferably massaged into wet hair for three minutes, then rinsed off with warm water, followed by a second, shorter shampoo and thorough rinsing. Preferably, hair is dried in a straight configuration such as by pulling long hair into one or more ponytails, or pinning short hair down with clips, or pressing short hair down with a scarf or netting.

### Example Formula 3

APG Straightening/Defrizzing Shampoo

| Ingredient | % by weight |
|---|---|
| APG C1214 (KW Chemicals, Hong Kong) | 98.5 |
| Fragrance | 1.5 |

The antibacterial properties of APG are sufficient to omit a bactericide in this formulation. Formulations according to this example are preferably used in a salon setting to assure that the product does not remain on the hair for an extended period of time, as the pH is not adjusted to 9.0.

### Example Formula 3A

APG Straightening/Defrizzing Shampoo

| Ingredient | % by weight |
|---|---|
| APG C1214 (KW Chemicals, Hong Kong) | 87.9 |
| Water | 10.4 |
| Fragrance | 01.7 |

The formulation according to Example 3 was diluted with water to decrease viscosity. Formulations according to this Example provided lathering attributes superior to those of Formula 3 and are also preferably used in a professional setting due to high pH. Surprisingly, both of formulations Example 3 and Example 3A shampoo are non-irritating to the scalp and do not result in hair breakage or dryness.

### Example Formula 4

High Foam APG Straightening/Defrizzing Shampoo Combined With or Added to a Base Shampoo Containing Sodium Laureth Sulfate

| Ingredient | % by weight |
|---|---|
| APG C1214 | 49.9 |
| Bullenairx Base Shampoo Formula 30-289A | 49.9 |
| Fragrance | 0.2 |

APG was added to an existing shampoo formulation, in this case one made by Bullenairx of Folcroft, Pa. The base shampoo used in this example was Bullenairx's Formula 30-289A, which contains about 50% sodium laureth sulfate as the foaming agent. No pH adjustment was necessary. Sodium lauryl/laureth sulfate could also be used as a separate ingredient, such as Standapol® ES-2, in place of the Bullenairx Base Shampoo.

### Example Formula 5

Straightening/Defrizzing Conditioning Shampoo with Color-Protectant (2000 ml)

| Ingredient | % by weight |
|---|---|
| Deionized water | 57.5 |
| SugaNate 160 UP | 37.1 |
| Merquat 2001 | 2.0 |
| Glucamate LT (rheology agent, Noveum) | 1.5 |
| Fragrance | 0.9 |
| Suttocide A | 0.5 |
| 20% aqueous acetic acid as pH adjuster | 0.5 |
| | 100% |

This example provides a richer, creamy feel during lathering and thicker, more appealing viscosity and color (off white, rather than pale yellow) by the addition of the rheology agent Glucamate® LT (Noveon, Cleveland, Ohio). Traditional rheology agents are not always compatible with APG formulations.

### Example Formula 6

Straightening/Defrizzing Styling Gel (2000 ml)

| Ingredient | % by weight |
|---|---|
| Deionized water | QS to 100% |
| APG (Plantaren ® 2000 N, Cognis) | 15.0 |
| Methylsulfonylmethane (SGS Research, Irvine, CA) | 6.9 |
| Xanthan Gum (CP Kelco) | 1.4 |
| Grapeseed Oil | 0.5 |
| Fragrance | 0.7 |
| Citric Acid Anhydrous Powder | QS to pH 8.0 |

Ingredients are blended in the above order and mixed for 25 minutes. In application, preferably a small amount of gel according to this formulation is applied with the palms and distributed evenly on wet or dry hair with a comb or brush, followed by air or heat-appliance drying and conventional styling.

In addition to curl relaxation, style retention is observed on all hair types after use of the shampoo or gel.

### Example Formula 7

Straightening/Defrizzing Cream Conditioner

| Ingredient | % by weight |
|---|---|
| APG SugaNate ® 160UP (Colonial) | 87.7 |
| PEG 400 Monostearate (Ruger) | 7.5 |
| Sugaquat ® Quaternium (Colonial) | 2.2 |
| Citric Acid Anhydrous Powder | 2.4 |
| Fragrance | 0.2 |
| | 100% |

This formulation may be used as a wash-out or leave-in conditioner, depending on the amount applied. In application as a leave-in straightener or defrizzer, preferably a small about of this cream is applied to wet or dry hair and distributed evenly with a comb or brush daily until desired straightness and smooth, non-frizzy hair texture is achieved. As a wash-out straightener or defrizzing agent the cream is preferably generously coated on clean, wet hair for 10 to 30 minutes, then rinsed out with warm water.

### Example Formula 8

Straightening/Defrizzing Wash-out Conditioner Detangler

| Ingredient | % by weight |
|---|---|
| APG SugaNate 160 UP (Colonial) | 76.0 |
| Peg 400 Monosteate (Ruger Chemical) | 8.0 |
| Glycerin | 5.0 |
| Glucamate LT (Noveon) | 5.0 |
| SugaQuat Quaternium (Colonial) | 3.0 |
| Citric acid anhydrous powder | 2.0 |
| Fragrance | 1.0 |
| | 100% |

The addition of glycerin in this example adds the benefit of detangling the hair after shampooing, while APGs reduce curl and frizz. This formulation is more viscous than the cream of Example 7, permitting easy dispensing, dispersal and wash-out after shampooing.

### Example Formula 9

Straightening/Defrizzing Hair Spray and Setting Solution (2000 ml)

| Ingredient | % by weight |
|---|---|
| Deionized Water | QS to 100% |
| APG SugaNate 160 UP | 15.0 |
| DL-Panthenol (American Chemical International) | 3.0 |
| Fragrance | 0.5 |
| Suttocide A | 0.5 |
| Citric Acid Anhydrous Powder | QS to pH of 9.0 |

Preferably the formulation is applied as a hair spray from a suitable spray container and applied to wet or dry hair prior to styling. As a setting solution, the spray is preferably applied more generously prior to setting hair on rollers or pulling hair straight with the use of elastics or clips.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A mammalian hair straightening composition comprising at least one alkyl polyglucoside in an amount of at least 15% by weight of the composition.

2. A composition according to claim 1 further comprising at least one further ingredient selected from the group comprising an antibacterial agent, a fragrance, a rheology agent, a conditioner, a pearlizing agent, a foam/viscosity stabilizer, a surfactant, a detangler, a foaming agent, a color protectant, an emollient, a processing aid, a dye, a plant oil, water and a pH adjuster.

3. A composition according to claim 1 comprising a shampoo.

4. A composition according to claim 1 comprising a hair conditioner.

5. A composition according to claim 1 comprising a hair gel.

6. A composition according to claim 1 comprising a hair spray.

7. A composition according to claim 1 comprising a hair setting composition.

8. A composition according to claim 1 said at least one alkyl polyglucoside comprising at least one of either sodium decylglucosides hydroxyproplylsulfonate or sodium laurylglucosides hydroxypropyl sulfonate.

9. A composition according to claim 3 comprising a washing or foaming base.

10. A composition according to claim 4 comprising d-panthenol or a polyquaternium.

11. A composition according to claim 1 having a pH in the range of from about 8.0 to about 11.5.

12. A composition according to claim 1 said at least one alkyl polyglucoside being present in an amount of at least 30% by weight of the composition.

13. A method for straightening mammalian hair comprising the step of applying to said hair a composition comprising at least one alkyl polyglucoside in an amount of at least 15% by weight of the composition.
